(19) United States
(12) Patent Application Publication
(10) Pub. No.: US 2016/0038035 A1
(43) Pub. Date: Feb. 11, 2016

(54) METHOD FOR ACQUIRING DYNAMIC INFORMATION OF LIVING BODY AND APPLICATIONS THEREOF

(71) Applicant: K-JUMP HEALTH CO., LTD., New Taipei City (TW)

(72) Inventor: CHAO MAN TSENG, New Taipei City (TW)

(21) Appl. No.: 14/451,939

(22) Filed: Aug. 5, 2014

(51) Int. Cl. A61B 5/01; A61B 5/00

(52) U.S. Cl. CPC A61B 5/01; A61B 5/0008; A61B 5/7278; A61B 5/6892; A61B 2560/0252; A61B 2562/0271; A61B 2560/029; A61B 2562/046

(57) ABSTRACT

A method for acquiring dynamic information of a living body comprises steps: arranging a plurality of temperature measurement points on a surface, and pointwise encoding positions of the plurality of temperature measurement points; decoding to receive the positions of the temperature measurement points and temperatures detected by the temperature measurement points in different time intervals with a same given length; obtaining an ambient temperature; comparing the temperatures of the temperature measurement points with the ambient temperature respectively to determine relative temperatures of the temperature measurement points; and comparing the relative temperature in the current time interval with the relative temperature in the last time interval for each of the temperature measurement points respectively to determine the temperature variations of the plurality of temperature measurement points in each time interval and thus acquire dynamic information of the living body on the surface.

### FIELD OF THE INVENTION

The present invention relates to an electronic detection method and the applications thereof, particularly to a method and device for monitoring a patient in real time.

### BACKGROUND OF THE INVENTION

The real time monitoring technologies includes the electrocardiograph monitor, the respiratory monitor, the blood pressure monitor, the electroencephalogram, the pulse monitor, the blood oxygen monitor, the carbon dioxide monitor, the body temperature monitor, the body weight meter, and various non-invasive medical auxiliary instruments. The medical-nursing field also eagerly anticipates a device able to real time monitor the postures and movements of aged persons and patients lying in beds and able to alarm the nursing personnel. Such a device is especially important for the patients risking epilepsy or a fall from the bed. Persistent monitoring the postures and movements of patients also favors grasping the health status and patients' reaction to medicine. Monitoring and analyzing sleep modes and physical activity levels would promote the quality of nursing aged persons and patients and decrease the probability of bedsore (pressure sore), especially for the aged persons and patients unable to move their bodies autonomously and lying in beds continuously.

The medical real time monitoring devices available currently are mainly the pressure-sensing air beds. The current air beds normally have only a single specification but apply to patients having different weights. For heavier patients, air pressure and air volume of the air bed should be increased lest the air bed be flattened. For lighter patients, air pressure and air volume of the air bed should be decreased lest the air bed be too hard and discomfort the patients. Anyhow, the air pressure of an air bed should be regulated according to the weight of the patient to achieve appropriate support force and provide comfort for the patient. Therefore, weighing the patient is very important for appropriately supplying air to an air bed. However, it is difficult to weigh the patients who are not ambulatory or unable to stand on a body weight meter. Thus, there are handicaps in regulating the air pressure of an air bed according to the weight of a patient. Therefore, the conventional technologies can not meet the market demand any more.

### SUMMARY OF THE INVENTION

The primary objective of the present invention is to provide a simple and precise method for monitoring a patient in real time to meet the market demand.

To achieve the abovementioned objective, the present invention proposes a method for acquiring dynamic information of a living body, which comprises steps: providing a plurality of temperature sensing elements contacting the surface of a living body and detecting the temperatures thereof, wherein the temperature sensing elements are distributed on a surface to form a temperature sensing unit; encoding the positions of the temperature sensing elements which are distributed at different positions on the surface of the temperature sensing unit; providing an ambient temperature sensor for detecting the ambient temperature; providing a timer; providing a controller triggered by the signal of the timer to undertake decoding to receive the information of the temperature sensing elements and the ambient temperature sensor; the controller decoding the positions of the temperature sensing elements, receiving the temperature information of the temperature sensing elements at different time intervals, and comparing the temperatures of the temperature sensing elements with the ambient temperature of the ambient temperature sensor to determine the temperature variations of the living body at different positions on the surface of the temperature sensing unit in different time intervals and thus acquire the dynamic information of the living body on the surface of the temperature sensing unit.

In some embodiments, the method of the present invention further comprises a step: providing a decoding processor, which decodes information of the temperature sensing elements and then transmitting the decoded information to the controller.

In some embodiments, encoding the positions of the temperature sensing elements includes steps of: providing a plurality of longitudinal axes and a plurality of transverse axes; undertaking a first encoding to encode the longitudinal axes and the transverse axes; arranging the temperature sensing elements at the intersections of the longitudinal axes and the transverse axes; using the result of the first encoding to undertake a second encoding to encode the positions of the temperature sensing elements on the temperature sensing unit.

In some embodiments, encoding the positions of the temperature sensing elements includes steps of: dividing the temperature sensing unit into a plurality of blocks each including part of the temperature sensing elements; undertaking a first encoding to encode the blocks; arranging the temperature sensing elements in the blocks; using the result of the first encoding to undertake a second encoding to encode the positions of the temperature sensing elements on the temperature sensing unit.

The present invention further proposes a method for acquiring dynamic information of a living body, which comprises steps of: arranging a plurality of temperature measurement points on a surface, and pointwise encoding the positions of the temperature measurement points; or alternatively arranging a plurality of temperature measurement points on a surface, dividing the surface into a plurality of blocks according to the temperature measurement points, and encoding the blocks to designate the positions of the temperature measurement points; decoding to receive the positions of the temperature measurement points on the surface and receiving the temperatures detected at the temperature measurement points in every time interval of a given length; and comparing the temperature detected by each temperature measurement point in the current time interval with the temperature detected by the same temperature measurement point in the last time interval to determine the temperature variation of each temperature measurement point in each time interval of a given length and thus acquire the dynamic information of the living body on the surface.

In some embodiments, the abovementioned method further comprises steps of: obtaining the ambient temperature simultaneously; comparing the temperatures of the temperature measurement points with the ambient temperature to determine the relative temperatures of the temperature measurement points; comparing the relative temperatures of the temperature measurement points in the current time interval with the relative temperatures of the same temperature measurement points in the last time interval to determine the temperature variations of the temperature measurement points in each time interval of a given length and thus acquire the dynamic information of the living body on the surface.

Another objective of the present invention is to provide an accurate and low-cost real time monitoring device to meet market demand.

To achieve the abovementioned objective, the present invention proposes a device for acquiring and converting dynamic information of a living body, which comprises a plurality of temperature sensing elements having been encoded and able to detect the surface temperature of a living body; a temperature sensing unit whose surface the temperature sensing elements are distributed on; an ambient temperature sensor detecting the ambient temperature; and an information processing unit connected with the ambient temperature sensor and all the temperature sensing elements to receive, process and convert the information of the ambient temperature sensor and all the temperature sensing elements. The information processing unit includes a timer; a controller triggered by the signal of the timer to calculate and convert the information of the ambient temperature sensor and all the temperature sensing elements into dynamic information of the living body; and an alarm receiving the dynamic information of the living body and generating sound, light, vibration, wireless signal or a combination thereof.

In some embodiments, the abovementioned temperature sensing elements are arranged on the surface of the temperature sensing unit in sequence.

In some embodiments, the temperature sensing unit includes a decoding processor decoding information of the temperature sensing elements and transmitting the information to the controller. In some embodiments, the alarm is a device selected from a group consisting of a buzzer, a display, a vibrator, a wireless signal transmitter, and the combinations thereof.

In some embodiments, the temperature sensing unit is a mattress including an air-permeable surface layer where the temperature sensing elements are attached; and a soft pad arranged inside the air-permeable surface layer, wherein the temperature sensing elements are interposed between the air-permeable surface layer and the soft pad.

Via the abovementioned technical schemes, the present invention outperforms the conventional technology in that the present invention can easily and cost-efficiently obtain the precise temperature variations of each of the temperature measurement points in every time interval of a given length and thus acquire the accurate dynamic information of a living body, merely disposing temperature measurement points on a surface. Therefore, the present invention can reduce the fabrication cost and meet market demand.

### BRIEF DESCRIPTION OF THE DRAWINGS

FIG. 1 is a block diagram schematically showing a first embodiment of the present invention;

FIG. 2 is a diagram schematically showing a first implementation of the temperature sensing unit according to FIG. 1;

FIG. 3 is a diagram schematically showing a second implementation of the temperature sensing unit according to FIG. 1;

FIG. 4 is a diagram schematically showing a third implementation of the temperature sensing unit according to FIG. 1;

FIG. 5 is a diagram schematically showing an application of the system shown according to FIG. 1;

FIG. 6 is a diagram schematically showing another application of the system shown according to FIG. 1; and

FIG. 7 is a block diagram schematically showing a second embodiment of the present invention.

### DETAILED DESCRIPTION OF THE PREFERRED EMBODIMENTS

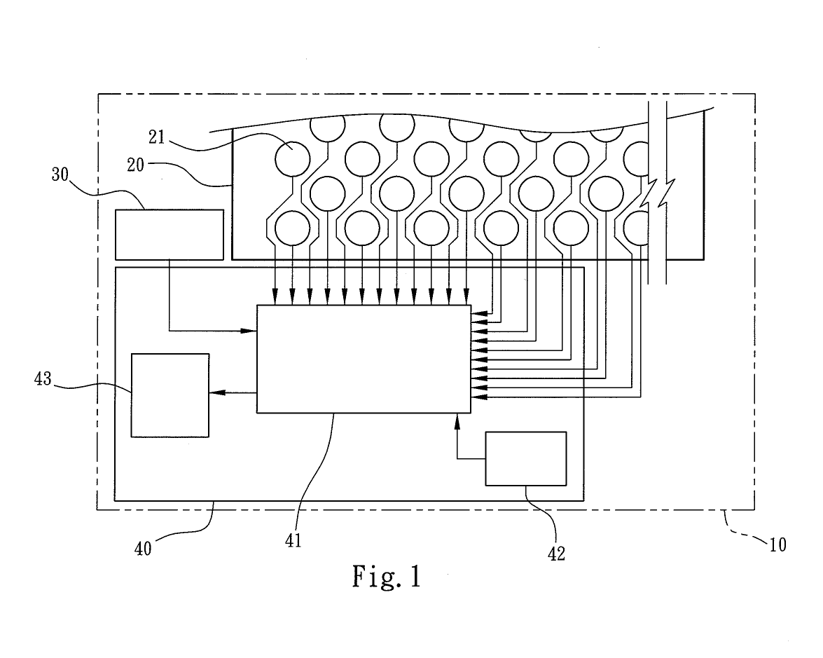

Below, embodiments will be used to demonstrate the technical contents of the present invention in cooperation with drawings. Refer to FIG. 1 a block diagram schematically showing a first embodiment of the present invention. The present invention discloses a method for acquiring dynamic information of a living body, which comprises steps: providing a plurality of temperature sensing elements 21 contacting the surface of a living body (such as a human body or a pet) and detecting the temperatures thereof, wherein it is preferred that the living body is a human body, and wherein the temperature sensing elements 21 are distributed on a surface to form a temperature sensing unit 20, and wherein it is preferred that the temperature sensing elements 21 are uniformly arranged on the surface of the temperature sensing unit 20 in sequence; encoding the positions of the temperature sensing elements 21 which are distributed at different positions on the surface of the temperature sensing unit 20; providing an ambient temperature sensor 30 for detecting an ambient temperature; providing a timer 42; and providing a controller 41 triggered by the signal of the timer 42 to undertake decoding to receive the information of the temperature sensing elements 21 and the ambient temperature sensor 30. Refer to FIG. 7 a block diagram schematically showing a second embodiment of the present invention. In the second embodiment, the method of the present invention comprises steps: decoding the temperature sensing elements 21 and transmitting the information to a decoding processor 24 of the controller 41; the controller 41 obtaining the information of the positions and temperatures of the temperature sensing elements 21 in different time intervals, and comparing the temperature information of the temperature sensing elements 21 with the temperature information of the ambient temperature sensor 30 (ambient temperature) to obtain the temperature variations of different positions of the temperature sensing unit 20 in different time intervals. Thereby is obtained the dynamic information of the living body on the temperature sensing unit 20. It should be noted: the present invention does not limit that the method of the present invention is only applicable to the system comprising the abovementioned elements and devices. The present invention further proposes a method for acquiring dynamic information of a living body, which comprises steps: arranging a plurality of temperature measurement points on a surface, and pointwise encoding the positions of the temperature measurement points; or alternatively arranging a plurality of temperature measurement points on a surface, dividing and encoding the surface into a plurality of blocks, and encoding the positions of the temperature measurement points according to the encoding of the blocks; decoding to receive the positions of the temperature measurement points on the surface and the temperature information of the temperature measurement points in each time interval of a given length; obtaining the current ambient temperature; comparing the temperatures of the temperature measurement points with the ambient temperature to determine the relative temperatures of the temperature measurement points with respect to the ambient temperature of the surface; and comparing the temperatures detected at the temperature measurement points in the current time interval with the temperatures detected at the same temperature measurement points in the last time interval to determine the temperature variations of the temperature measurement points in each time interval of a given length and thus acquire the dynamic information of the living body on the surface.

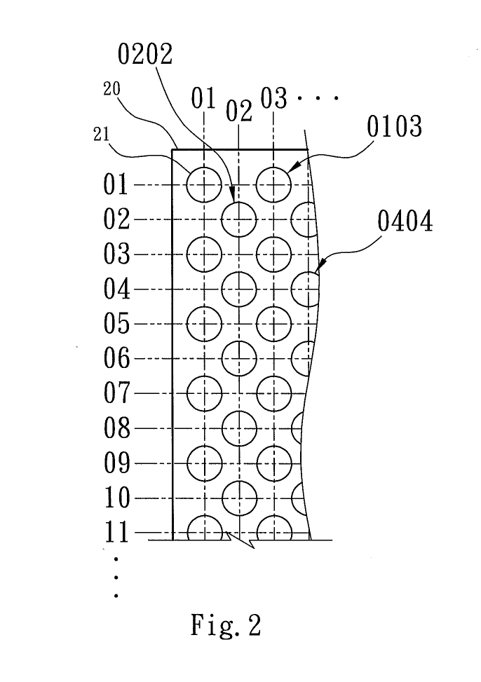

Below, three implementations are used to further demonstrate the method of encoding the positions of the temperature sensing elements 21. Refer to FIG. 2 a diagram schematically showing a first implementation of the temperature sensing unit 20 in FIG. 1. A first encoding is to designate a plurality of transverse axes with the serial numbers 01, 02, 03, 04, 05, 06, 07, 08, 09, 10, 11, - - - , and designate a plurality of longitudinal axes with the serial numbers 01, 02, 03, - - - . The temperature sensing elements 21 are respectively arranged at the intersections of the longitudinal axes and the transverse axes. The second encoding is to sequentially assign the temperature sensing elements 21 with the serial numbers of the transverse axes and the serial numbers of the longitudinal axes of the intersections where the temperature sensing elements 21 are located. For example, the first encoding respectively assigns the serial numbers 01, 02, 03, 04, 05, 06, 07, 08, 09, 10, 11, - - - and the serial numbers 01, 02, 03, - - - to the transverse axes and the longitudinal axes; the second encoding respectively assigns the serial numbers 02, 01 and 04 of the transverse axes and the serial numbers 02, 03, and 04 of the longitudinal axes to three temperature sensing elements 21, which are separately located at the intersections of the transverse axes designated by the serial numbers 02, 01 and 04 and the longitudinal axes designated by the serial numbers 02, 03, and 04. Thereby, the three temperature sensing elements 21 are respectively encoded with the codes 0202, 0103 and 0404. Thus, the controller 41 (shown in FIG. 1) can learn the positions where the temperature sensing elements 21 are located on the surface of the temperature sensing unit 20 according to the codes thereof.

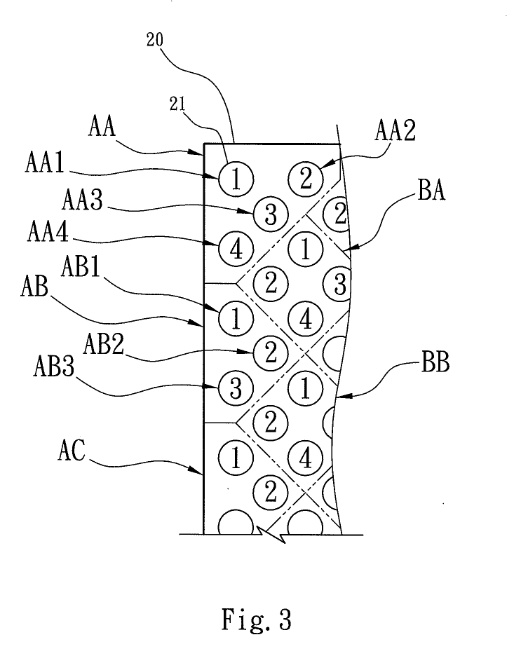

Refer to FIG. 3 a diagram schematically showing a second implementation of the temperature sensing unit 20 in FIG. 1. In FIG. 3, the temperature sensing unit 20 is divided into a plurality of blocks. The first encoding is to sequentially encode the blocks with AA-AZ, BA-BZ, - - - . The second encoding is to add serial numbers to the temperature sensing elements 21 inside each block. For one example, four temperature sensing elements 21 are located inside the block AA, and the second encoding respectively adds serial numbers 1, 2, 3 and 4 to the four temperature sensing elements 21 and designates the four temperature sensing elements 21 with AA1, AA2, AA3 and AA4. For another example, three temperature sensing elements 21 are located inside the block AB, and the second encoding respectively adds serial numbers 1, 2 and 3 to the three temperature sensing elements 21 and designates the three temperature sensing elements 21 with AB1, AB2 and AB3. Then, the controller 41 (shown in FIG. 1) can learn the positions where the temperature sensing elements 21 are located on the surface of the temperature sensing unit 20 according to the codes thereof.

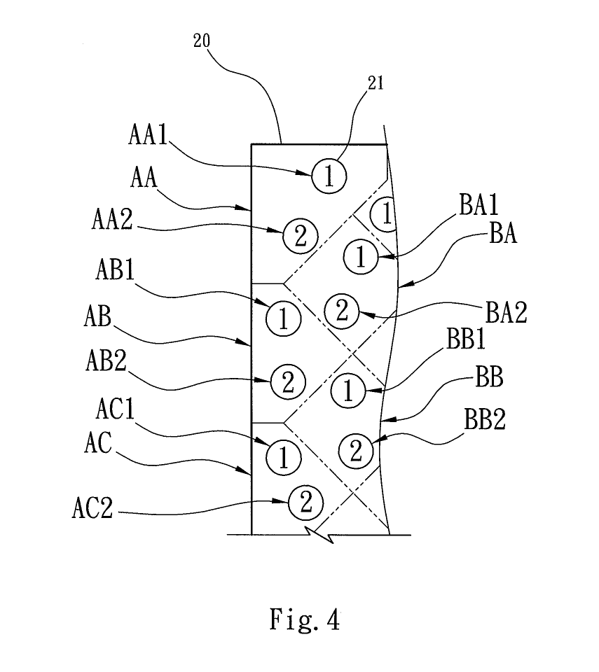

Refer to FIG. 4 a diagram schematically showing a third implementation of the temperature sensing unit 20 in FIG. 1. Similarly to the second implementation, the temperature sensing unit 20 is also divided into a plurality of blocks in FIG. 4. The third implementation is different from the second implementation in that the temperature sensing elements 21 are not uniformly but irregularly distributed on the temperature sensing unit 20. However, the temperature sensing elements 21 are still separated by appropriate distances in FIG. 4. As shown in FIG. 4, each of the blocks AA, AB, AC, BA and BB only have two temperature sensing elements 21 after the first encoding. The second encoding adds serial numbers to the temperature sensing elements 21 inside each block. Thus, the temperature sensing elements 21 located inside the blocks AA, AB, AC, BA and BB are respectively encoded with the codes AA1, AA2, AB1, AB2, AC1, AC2, BA1, BA2, BB1 and BB2. Then, the controller 41 (shown in FIG. 1) can learn the positions where the temperature sensing elements 21 are located on the surface of the temperature sensing unit 20 according to the codes thereof.

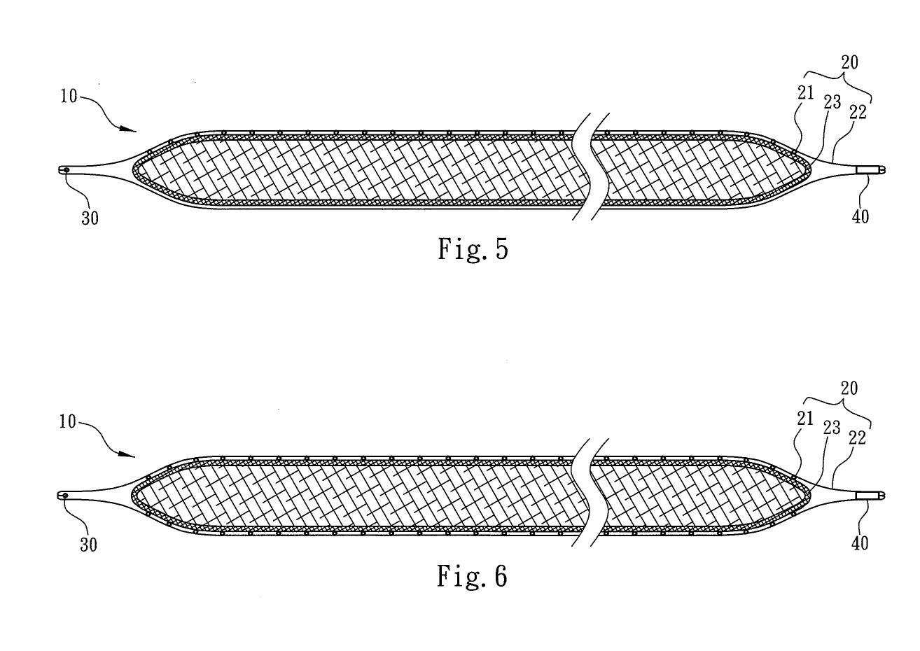

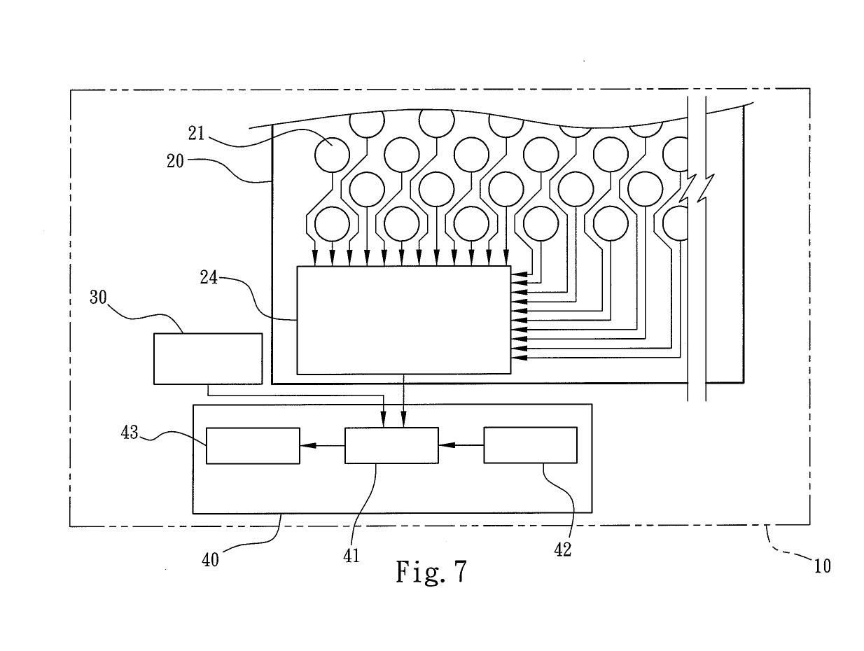

The present invention also proposes a device for acquiring and converting dynamic information of a living body. Refer to FIG. 1 and FIG. 5. FIG. 5 is a diagram schematically showing an application of the system shown in FIG. 1. The device 10 of the present invention comprises a plurality of temperature sensing elements 21 having been encoded and able to detect the temperatures of the surface of a living body; a temperature sensing unit 20 formed by distributing the temperature sensing elements 21 on the surface thereof, wherein the temperature sensing elements 21 can be uniformly arranged on the surface of the temperature sensing unit 20 in sequence; an ambient temperature sensor 30 detecting the ambient temperature; and an information processing unit 40 connected with the ambient temperature sensor 30 and the temperature sensing elements 21 to receive, process and convert the information of the ambient temperature sensor 30 and the temperature sensing elements 21. The information processing unit 40 includes a timer 42; a controller 41 triggered by the signal of the timer 42 to calculate and convert the information of the ambient temperature sensor 30 and the temperature sensing elements 21 into dynamic information of the living body; and an alarm 43 receiving the dynamic information of the living body and generating sound, light, vibration, wireless signal or combinations thereof. In some embodiments, the alarm 43 is a device selected from a group consisting of a buzzer, a display, a vibrator, a wireless signal transmitter, and the combinations thereof. In the second embodiment shown in FIG. 7, the temperature sensing unit 20 includes a decoding processor 24 decoding the information of the temperature sensing elements 21 and transmitting information to the controller 41. If the living body is a human body, especially a human body of an aged person, the temperature sensing unit 20 of the device 10 may be in form of a mattress, a bed sheet, a seat cushion, or a pillow. A mattress is used to exemplify the temperature sensing unit 20 below. The temperature sensing unit 20 (mattress) includes an air-permeable surface layer 22 where the temperature sensing elements 21 are attached; and a soft pad 23 arranged inside the air-permeable surface layer 22, wherein the temperature sensing elements 21 are interposed between the air-permeable surface layer 22 and the soft pad 23. In some embodiments, the ambient temperature sensor 30 and the information processing unit 40 are sewn on the perimeter of the mattress (the temperature sensing unit 20). In some embodiments, the temperature sensing elements 21 are arranged on both sides of the mattress, as shown in FIG. 6. While a human body lies on the mattress (the temperature sensing unit 20), the human body would press against some of the temperature sensing elements 21. Thus, the temperature sensing elements 21 that are pressed against by the human body will detect the surface temperature of the human body. Meanwhile, the ambient temperature sensor 30 also detects the ambient temperature. While the timer 42 periodically sends signals to the controller 41, the controller 41 immediately decodes the information of the positions and temperatures of the temperature sensing elements 21 on the mattress (the temperature sensing unit 20), as shown in FIG. 1. Alternatively, the controller 41 lets the decoding processor 24 decode the information of positions and temperatures of the temperature sensing elements 21 on the mattress (the temperature sensing unit 20); meanwhile the controller 41 compares the temperatures of the temperature sensing elements 21 with the ambient temperature of the ambient temperature sensor 30 and generates the temperature differences thereof; the controller 41 also compares the current temperature differences and the last temperature differences to obtain the dynamic information of the human body. In other words, the temperature detected by a specified temperature sensing element 21 is compared with the ambient temperature detected by the ambient temperature sensor 30 to determine the temperature difference at the location of the specified temperature sensing element 21. If the temperature difference of the specified temperature sensing element 21 is greater and the temperature of the specified temperature sensing element 21 is higher, it indicates that the human body is exactly compressing the specified temperature sensing element 21; we can learn the compressed area via decoding the position of the specified temperature sensing element 21. At this time, the controller 41 also compares the current temperature difference with the last temperature difference or the next temperature difference to obtain the time-domain temperature difference variation. If the temperature difference variation is greater and the temperature is lower, it indicates that the human body has left the position of the specified temperature sensing element 21. If the temperature difference variation is smaller, it indicates that the human body has kept immobile and persistently compressed the specified temperature sensing element 21 for a period of time. If the period of time has exceeded the preset length of time, it means that the human body has compressed the identical temperature sensing element 21 for too long a time. Thus, the alarm 43 alerts the nursing personnel to consider whether to turn the body over or move the body lest necrosis or bedsore occur. The alarm 43 may be a buzzer generating warning sounds, a display presenting text or flash, a vibrator generation vibrations, or a wireless signal transmitter sending information to a handheld communication device. While there is no great temperature difference between the temperatures, which are detected by an identical temperature sensing element 21 periodically, and the ambient temperatures, which are detected by the ambient temperature sensor 30 at the same time, it indicates that the human body does not compress the temperature sensing element 21. While the temperatures, which are respectively detected by all the temperature sensing elements 21 periodically, are near the ambient temperatures, which are detected by the ambient temperature sensor 30 at the same time, it indicates that the human body has left the mattress (the temperature sensing unit 20). In such a case, the alarm 43 will generate an alert to remind the nursing personnel to check whether the aged patient fell from the bed or got out of the bed by himself. Therefore, the present invention can provide instant care for patients and aged persons and promote the quality of nursing.

The present invention has been described in detail with the embodiments above. However, these embodiments are only to exemplify the present invention but not to limit the scope of the present invention. Any equivalent modification or variation according to the spirit of the present invention is to be also included within the scope of the present invention.

What is claimed is:

1. A method for acquiring dynamic information of a living body, comprising steps of:
providing a plurality of temperature sensing elements contacting surface of a living body and detecting temperatures of the surface of the living body, wherein the temperature sensing elements are distributed on a surface to form a temperature sensing unit;
encoding positions of the plurality of temperature sensing elements which are distributed at different positions on the surface of the temperature sensing unit;
providing an ambient temperature sensor for detecting an ambient temperature;
providing a timer; and
providing a controller triggered by a signal of the timer to undertake decoding to receive information of the plurality of temperature sensing elements and the ambient temperature sensor;
wherein the controller obtains information of positions of all the temperature sensing elements and temperatures detected by all the temperature sensing elements in different time intervals and compares the temperatures detected by all the temperature sensing elements with the ambient temperature detected by the ambient temperature sensor in the different time intervals to determine temperature variations of the living body at different positions of the temperature sensing unit in the different time intervals and thus acquire dynamic information of the living body on the surface of the temperature sensing unit.

2. The method for acquiring dynamic information of a living body according to claim 1 further comprising a step: providing a decoding processor decoding information of the plurality of temperature sensing elements and transmitting the information to the controller.

3. The method for acquiring dynamic information of a living body according to claim 1, wherein encoding positions of the plurality of temperature sensing elements includes steps of: arranging the temperature sensing elements at intersections of a plurality of longitudinal axes and a plurality of transverse axes distributed on the surface of the temperature sensing unit; undertaking a first encoding to encode the longitudinal axes and the transverse axes; and using a result of the first encoding to undertake a second encoding to encode the positions of the temperature sensing elements on the surface of the temperature sensing unit.

4. The method for acquiring dynamic information of a living body according to claim 2, wherein encoding positions of plurality of the temperature sensing elements includes steps of: arranging the temperature sensing elements at intersections of a plurality of longitudinal axes and a plurality of transverse axes distributed on the surface of the temperature sensing unit; undertaking a first encoding to encode the longitudinal axes and the transverse axes; and using a result of the first encoding to undertake a second encoding to encode the positions of the temperature sensing elements on the surface of the temperature sensing unit.

5. The method for acquiring dynamic information of a living body according to claim 1, wherein encoding positions of the plurality of temperature sensing elements includes steps of: dividing the temperature sensing unit into a plurality of blocks each including part of the temperature sensing elements; undertaking a first encoding to encode the plurality of blocks; using a result of the first encoding to undertake a second encoding to encode the positions of the temperature sensing elements on the surface of the temperature sensing unit.

6. The method for acquiring dynamic information of a living body according to claim 2, wherein encoding positions of the plurality of temperature sensing elements includes steps of: dividing the temperature sensing unit into a plurality of blocks each including the temperature sensing elements; undertaking a first encoding to encode the plurality of blocks; using a result of the first encoding to undertake a second encoding to encode the positions of the temperature sensing elements on the surface of the temperature sensing unit.

7. A method for acquiring dynamic information of a living body, comprising steps of:
arranging a plurality of temperature measurement points on a surface, and encoding positions of the plurality of temperature measurement points; decoding to receive the positions of the plurality of temperature measurement points on the surface and temperatures detected by the plurality of temperature measurement points in different time intervals a same given length; and
comparing the current temperature with the last temperature for each of the plurality of temperature measurement points to determine temperature variation of each of the plurality of temperature measurement points in each time interval and thus acquire dynamic information of the living body on the surface.

8. The method for acquiring dynamic information of a living body according to claim 7 further comprising steps of: obtaining an ambient temperature; comparing the temperatures of the plurality of temperature measurement points with the ambient temperature respectively to determine relative temperatures of the plurality of temperature measurement points; and comparing the relative temperatures in the current time interval with the relative temperatures in the last time interval for each of the plurality of temperature measurement points respectively to determine the temperature variations of the of the plurality of temperature measurement points in each time interval and thus acquire dynamic information of the living body on the surface.

9. A device for acquiring and converting dynamic information of a living body, comprising
a plurality of temperature sensing elements having been encoded and able to detect temperatures of surface of a living body;
a temperature sensing unit formed by distributing the plurality of temperature sensing elements on a surface thereof;
an ambient temperature sensor detecting an ambient temperature; and
an information processing unit connected with the ambient temperature sensor and the plurality of temperature sensing elements to receive, process and convert information of the ambient temperature sensor and the plurality of temperature sensing elements, the information processing unit including
a timer;
a controller triggered by a signal of the timer to calculate and convert information of the ambient temperature sensor and the plurality of temperature sensing elements into dynamic information of the living body; and
an alarm receiving the dynamic information of the living body and generating sound, light, vibration, wireless signal or combinations thereof.

10. The device for acquiring and converting dynamic information of a living body according to claim 9, wherein the plurality of temperature sensing elements are arranged on the surface of the temperature sensing unit in sequence.

11. The device for acquiring and converting dynamic information of a living body according to claim 9, wherein the temperature sensing unit includes a decoding processor decoding information of the plurality of temperature sensing elements and transmitting the information to the controller, and wherein the alarm is a device selected from a group consisting of a buzzer, a display, a vibrator, a wireless signal transmitter, and combinations thereof.

12. The device for acquiring and converting dynamic information of a living body according to claim 10, wherein the temperature sensing unit includes a decoding processor decoding information of the plurality of temperature sensing elements and transmitting the information to the controller, and wherein the alarm is a device selected from a group consisting of a buzzer, a display, a vibrator, a wireless signal transmitter, and combinations thereof.

13. The device for acquiring and converting dynamic information of a living body according to claim 12, wherein the temperature sensing unit is in form of a mattress, and wherein the temperature sensing unit includes an air-permeable surface layer where the plurality of temperature sensing elements are attached, and a soft pad arranged inside the air-permeable surface layer, wherein the plurality of temperature sensing elements are interposed between the air-permeable surface layer and the soft pad.
